(19) United States
(12) Patent Application Publication
(10) Pub. No.: US 2011/0160636 A1
(43) Pub. Date: Jun. 30, 2011

(54) DEVICE AND METHOD FOR INHIBITING COMPLEMENT ACTIVATION

(76) Inventor: Rekha Bansal, Twinsburg, OH (US)

(21) Appl. No.: 13/063,301

(22) PCT Filed: Sep. 10, 2009
(86) PCT No.: PCT/US09/56524

§ 371 Date: Mar. 10, 2011

(30) Foreign Application Priority Data
Sep. 10, 2008 (US) 61/095822

(51) Int. Cl. A61M 1/34

(52) U.S. Cl. 604/6.09

(57) ABSTRACT

An extracorporeal device for inhibiting alternative complement pathway activation includes a support structure an anti-complement antibody disposed on or within the support structure and a first conduit for conducting blood of the subject to the anti-complement antibody. The anti-complement antibody can bind to the complement protein and remove the complement protein from the blood.

### RELATED APPLICATION

This application claims priority from U.S. Provisional Application No. 61/095,822, filed Sep. 10, 2008, the subject matter, which is incorporated herein by reference.

### FIELD OF THE INVENTION

The present invention relates to complement activation. Particularly, the present invention relates to a method for inhibiting complement activation via an extracorporeal device.

### BACKGROUND OF THE INVENTION

It is estimated that approximately twenty million procedures involving extracorporeal blood circulation are performed annually. Approximately 10,000 renal transplantations are performed annually in the U.S. along with about 500,000 open-heart surgeries, both procedures requiring extracorporeal blood treatment. In addition, approximately ninety thousand plasmapheresis procedures are carried out annually in the U.S. Blood oxygenation has been used to treat patients suffering from acute respiratory failure and infants with diaphragmatic hernia. It is likely that new procedures, which are in development, such as the implantation of artificial hearts and artificial livers, will increase the use of extracorporeal circulation.

Complement pathway is activated when blood comes in contact with an artificial surface of an extracorporeal device. As a result of initial trigger, the cascade of events start and progress into forming anaphylatoxins and finally activating leukocytes, lymphocytes, and platelets. All these cell types are known to have receptors for anaphylatoxins, which cause cellular activation leading to pathological consequences. In complement cascade, the native C3 is converted into C3b and C3a. The newly formed C3b interacts with properdin and factor B to form the C3 convertase, which cleaves additional C3 molecules into C3b and C3a and C5 molecules into C5b and C5a. Both C3a and C5a are potent anaphylatoxins. C5b deposits onto cell surfaces and can initiate the formation of C5b-9 complexes. Neutrophils, monocytes and platelets have receptors for anaphylatoxins C3a and C5a and therefore effectively activates these cell types. T lymphocytes and mast cells can also be activated by anaphylatoxins.

Several antibodies have been developed against C3, C3b, B, Ba, Bb, P, D, C5, C6, C7, C8, and C9. These monoclonal or polyclonal antibodies specifically bind the lited proteins with high affinity. Some of these antibodies have been used to detect the presence of proteins in blood/tissue/cell cultures using ELISA, western blots, and immuno-staining methods. Additional studies have used these antibodies as therapeutic antibodies where, the antibody is injected into the mammal for obtaining beneficial effects. In the latter case, the antibody is categorized as a drug therapeutic. Such therapeutics antibodies neutralize a particular protein of interest without affecting the concentration of the target protein in the blood. As a result, the antigen-antibody complex remains in the body and is removed by the system via normal routes.

### SUMMARY OF THE INVENTION

The present invention relates to an extracorporeal device for inhibiting alternative complement pathway activation. The extracorporeal device includes a support structure, an anti-complement antibody disposed on or within the support structure, and a first conduit for conducting blood to the anti-complement antibody. The anti-complement antibody can bind to complement protein and remove the complement protein from the blood. Binding and removing the complement protein from the blood of a subject can inhibit the anti-complement alternative complement pathway activation in the blood of the subject.

In an aspect of the invention, the support structure can include a matrix that comprises at least one of agarose, cellulose, dextrin, polystyrene, polyethersulfone, polyvinyl difluoride, ethylene vinyl alcohol, polycarbonate, polyether, polyether carbonate, regenerated cellulose, cellulose acetate, polylactic acid, nylon, or polyurethane. The anti-complement antibody can be coated on the support structure.

The antibody can include at least one of an anti-C3 antibody, anti-C3b antibody, anti-Ba antibody, anti-Bb antibody, anti-P antibody, anti-D antibody, anti-C5 antibody, anti-C5a antibody, anti-C6 antibody, anti-C7 antibody, anti-C8 antibody, and anti-C9 antibody. The antibody can be raised in a mammal. The antibody can also be monoclonal, polyclonal, recombinant, monospecific, bispecific, dimeric, humanized, chimeric, single chain, human, bispecific, truncated or mutated. In an aspect of the invention, the antibody can be an IgG, F(ab′)2, F(ab)2, Fab′, Fab, scFv, truncated IgG, or recombinant antibody.

In another aspect of the invention, the blood contacted with the anti-complement antibody is incapable of activating the alternative complement pathway when returned to the subject. The removal of the complement protein in the blood prevents activation of neutrophils, monocytes, basophils, lymphocytes, and platelets via the alternative pathway.

In a further aspect of the invention, the anti-complement antibody can reduce the level of properdin in the blood. The reduced levels of properdin in blood can decrease levels of C3a, C5a, Bb, C5b-9 as a result of decreased alternative complement pathway activation during extracorporeal circulation. The reduced levels of properdin can also reduce cellular activation in blood from the subject following extracorporeal circulation.

In yet another aspect of the invention, the device can include a second conduit for returning blood to the subject. The complement protein is removed from the blood returned to the subject.

In a further aspect of the invention, the anti-complement antibody can be covalently adhered to a biocompatible polymer matrix. The polymer matrix can be in the form of a membrane. The support structure can also include the particulate polymer matrix. The particulate polymer matrix can have reactive groups, such as an aldehyde, hydroxyl, thiol, carboxyl and/or amino groups that are capable of reacting with the antibody to adhere the antibody to the matrix.

In another aspect of the invention, the extracorporeal device can be coupled to at least one of an artificial heart-lung device or a hemodialysis unit such that blood flows through both the artificial heart lung device or hemodialysis unit and the extracorporeal device.

The present invention also relates to a method of inhibiting alternative complement pathway activation in a subject. The method includes passing a bodily fluid of the subject through an extracorporeal device. The device can include a support structure, an anti-complement antibody disposed on or within the support structure, and a first conduit for conducting bodily fluid of the subject to the anti-complement antibody. The anti-complement antibody can bind to and remove complement protein in the bodily fluid. The bodily fluid contacted with anti-complement antibody can then returned to the subject. In an aspect of the invention, the bodily fluid can comprise whole human blood.

In another aspect of the invention, the support structure can include a matrix that comprises at least one of agarose, cellulose, dextrin, polystyrene, polyethersulfone, polyvinyl difluoride, ethylene vinyl alcohol, polycarbonate, polyether, polyether carbonate, regenerated cellulose, cellulose acetate, polylactic acid, nylon, or polyurethane. The anti-complement antibody can be coated on the support structure.

The antibody can include at least one of an anti-C3 antibody, anti-C3b antibody, anti-Ba antibody, anti-Bb antibody, anti-P antibody, anti-D antibody, anti-C5 antibody, anti-C5a antibody, anti-C6 antibody, anti-C7 antibody, anti-C8 antibody, and anti-C9 antibody. The antibody can be raised in a mammal. The antibody can also be monoclonal, polyclonal, recombinant, monospecific, bispecific, dimeric, humanized, chimeric, single chain, human, bispecific, truncated or mutated. In an aspect of the invention, the antibody can be an IgG, F(ab′)2, F(ab)2, Fab′, Fab, scFv, truncated IgG, or recombinant antibody.

In another aspect of the invention, the blood contacted with the anti-complement antibody is incapable of activating the alternative complement pathway when returned to the subject. The removal of the complement protein in the blood prevents activation of neutrophils, monocytes, basophils, lymphocytes, and platelets via the alternative pathway.

In a further aspect of the invention, the anti-complement antibody can reduce the level of properdin in the blood. The reduced levels of properdin in blood can decrease levels of C3a, C5a, Bb, C5b-9 as a result of decreased alternative complement pathway activation during extracorporeal circulation. The reduced levels of properdin can also reduce cellular activation in blood from the subject following extracorporeal circulation.

In yet another aspect of the invention, the device can include a second conduit for returning blood to the subject. The complement protein is removed from the returned blood.

In a further aspect of the invention, the anti-complement antibody can be covalently adhered to a biocompatible polymer matrix. The polymer matrix can be in the form of a membrane. The support structure can also include a particulate polymer matrix. The particulate polymer matrix can have reactive groups, such as an aldehyde, hydroxyl, thiol, carboxyl and/or amino groups that are capable of reacting with the antibody to adhere the antibody to the matrix.

The present invention also relates to an extracorporeal system for inhibiting alternative complement pathway activation in a subject. The system includes a support structure, an anti-complement antibody disposed on or within the support structure, a first conduit for conducting blood of a subject to the anti-complement antibody, and a second conduit for returning blood contacted with anti-complement antibody to the subject. The anti-complement antibody can bind to and remove complement protein in the blood.

In an aspect of the invention, the support structure can include a matrix that comprises at least one of agarose, cellulose, dextrin, polystyrene, polyethersulfone, polyvinyl difluoride, ethylene vinyl alcohol, polycarbonate, polyether, polyether carbonate, regenerated cellulose, cellulose acetate, polylactic acid, nylon, or polyurethane. The anti-complement antibody can be coated on the support structure.

The antibody can include at least one of an anti-C3 antibody, anti-C3b antibody, anti-Ba antibody, anti-Bb antibody, anti-P antibody, anti-D antibody, anti-C5 antibody, anti-C5a antibody, anti-C6 antibody, anti-C7 antibody, anti-C8 antibody, and anti-C9 antibody. The antibody can be raised in a mammal. The antibody can also be monoclonal, polyclonal, recombinant, monospecific, bispecific, dimeric, humanized, chimeric, single chain, human, bispecific, truncated or mutated. In an aspect of the invention, the antibody can be an IgG, F(ab′)2, F(ab)2, Fab′, Fab, scFv, truncated IgG, or recombinant antibody.

In another aspect of the invention, the blood contacted with the anti-complement antibody is incapable of activating the alternative complement pathway when returned to the subject. The removal of the complement protein in the blood prevents activation of neutrophils, monocytes, basophils, lymphocytes, and platelets via the alternative pathway.

In a further aspect of the invention, the anti-complement antibody can reduce the level of properdin in the blood. The reduced levels of properdin in blood can decrease levels of C3a, C5a, Bb, C5b-9 as a result of decreased alternative complement pathway activation during extracorporeal circulation. The reduced levels of properdin can also reduce cellular activation in blood from the subject following extracorporeal circulation.

In a further aspect of the invention, the anti-complement antibody can be covalently adhered to a biocompatible polymer matrix. The polymer matrix can be in the form of a membrane. The support structure can also include a particulate polymer matrix. The particulate polymer matrix can have reactive groups, such as an aldehyde, hydroxyl, thiol, carboxyl and/or amino groups that are capable of reacting with the antibody to adhere the antibody to the matrix.

In another aspect of the invention, the extracorporeal system can be coupled with at least one of an artificial heart-lung device or a hemodialysis unit such that blood flows through both the artificial heart lung device or hemodialysis unit and the extracorporeal device.

### BRIEF DESCRIPTION OF THE DRAWINGS

FIG. 1 illustrates a properdin trimer attached to non-blocking and blocking anti-properdin monoclonal antibodies.

FIG. 2 illustrates a round bead matrix, which is coated with protein-G. The monoclonal antibody MoAb⁷¹⁻¹¹⁰ was conjugated to the matrix in a fully oriented form with binding regions exposed because protein-G is capable of orienting the monoclonal antibody. Also shown is the blood sample. Triangles represent the properdin molecules. Properdin from blood binds the antibody. The bound properdin can be eluted off the column.

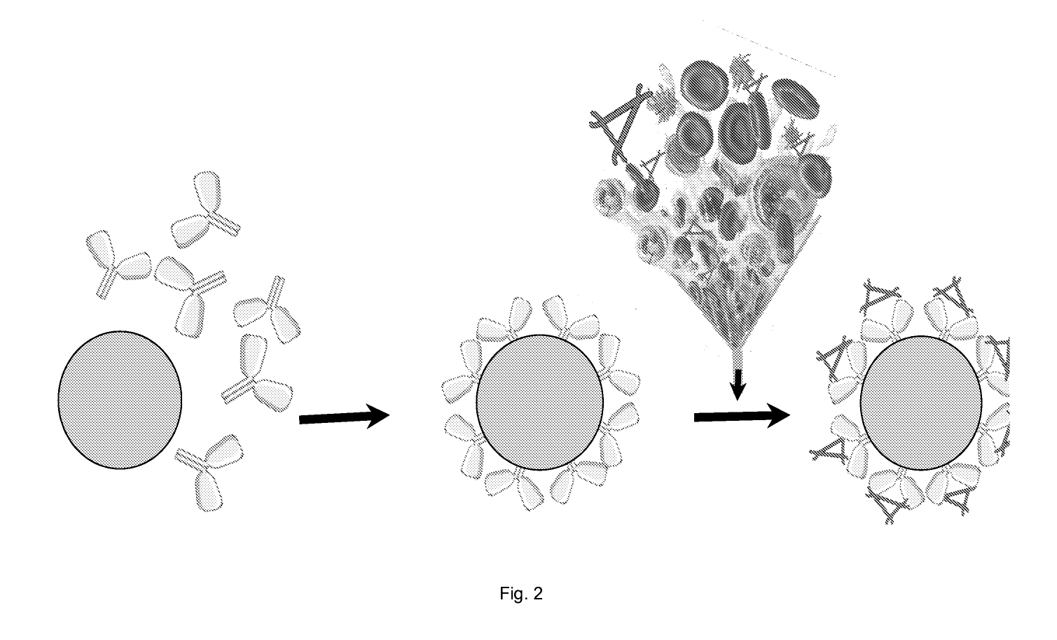

FIG. 3 illustrates a device in accordance with the present invention before and after the blood has passed through. Properdin bound beads are shown in zoom.

FIG. 4 illustrates the blood with properdin being introduced into the “device”. As shown the blood coming out from the other end is free of properdin. The arrow indicates that such blood would be deficient in alternative complement pathway (AP) activation.

FIG. 5 compares the AP dependent hemolysis of human serum eluted off the three columns that differ in the way monoclonal antibody⁷¹⁻¹¹⁰ is bound to the matrix. Three columns were prepared. The first column has sepharose beads 300 microns with monoclonal antibody⁷¹⁻¹¹⁰ covalently linked via the linker chemistry. The second bead column contain the F(ab′)2 fragment of the monoclonal antibody. In the third case, the beads are smaller in size—160 microns. These beads were coated with covalently bound protein-G. The monoclonal antibody was cross linked to the protein G to acquire the correct orientation onto the bead. As shown, the first two columns did not bind properdin and the serum is capable of AP activation. In the third column, the serum resists AP activation because of lack of properdin on the monoclonal antibody. The inhibition of AP activation in the column was comparable to the sample in which the monoclonal antibody was added to the control serum at 5 μg/ml. The comparable lack of AP activation in serum from the third column and that of the positive control suggests that the device may function as proposed in this invention.

FIG. 6 illustrates repetition of the experiment in FIG. 5 but by using the third column A small 2 ml column was set up and nearly 10 ml of the human serum was passed over the column to allow properdin depletion from serum. Fractions of 1 ml size were collected and assayed using hemolysis assay. As shown, all five fractions inhibited AP activation primarily due to the loss of properdin into the column. This study further confirms that at least 10 ml of serum can be passed through the 2 ml column, which roughly translates into 20 ml of whole blood. These serum samples were assayed for the presence of properdin by using C3b-P binding assay shown in FIG. 7. The serum samples were also tested for factor B levels as shown in FIG. 8

FIG. 7 illustrates the absence of properdin in fractions that demonstrated lack of AP activation. Both positive and negative controls showed appropriate values. The monoclonal antibody added to control serum prevents complement activation and hence prevents properdin binding to C3b. the presence of free properdin in all fractions was measured.

FIG. 8 illustrates the presence of factor B to the same levels in all samples clearly suggesting that the device in accordance with the present invention does not affect the levels of factor B.

FIG. 9 illustrates the evaluation of efficacy of the 2 ml column. This experiment is a repeat of the previous experiment but with larger volume of human serum (82 ml). Nearly 100 ml of human serum was introduced into the cartridge at a flow rate of 1 ml/minute. Fractions of 1 ml size were collected over the course of 2 hours. Each fraction was tested for AP activity and properdin (FIG. 10) levels. Measurement of AP activity is shown in this Figure. As shown, fraction #82 corresponds to the 82 ml of human serum lacks the AP activity. However, the AP activity appear to return at fractions around 85-88. Serum control with full activity is shown. Fractions near 100 or greater display the same lysis kinetic.

FIG. 10 illustrates the levels of properdin in column fractions.

FIG. 11 illustrates the results of the experiment performed in FIG. 9. The column following serum elution was extensively washed with PBS and eluted with elution buffer. To determine if properdin was the sole player in making serum inactive towards AP activation, the bound proteins were eluted off the column. The eluted material was subjected to 6-18% gradient SDS-PAGE. The blots were prepared and stained with anti-P, anti-B and only secondary antibody. As shown, there is no factor D or the artifact as the third blot showed no band in the absence of any primary antibody. However, the first blot shows properdin bands near 50K region.

### DETAILED DESCRIPTION

The present invention provides a method of making and using an extracorporeal device for removing a complement protein from bodily fluids of a subject so that the bodily fluid loses the ability to activate complement pathways. The bodily fluid is not limited to blood, but can include other bodily fluids, such as plasma and serum. Removal of complement proteins is performed using antigen-antibody interactions. Anti-complement antibodies are immobilized onto a solid support structure of the extracorporeal device and a bodily fluid, such as blood, can be passed through the device. Contacting a bodily fluid, such as blood, with the anti-complement antibody of the extracorporeal device can cause a complete/partial depletion of a target complement protein from the bodily fluid and can reduce in the subject or patient being treated: alternative complement pathway activation, complexes of antigen and antibody, levels of C3a/C5a (compared to C3a/C5a levels present in a subject or patient at the start of the extracorporeal procedure) the ability to make C5b-9 complexes, the levels of complement dependent activation of neutrophils, monocytes, and platelets, the levels of cytokines, TNF alpha, and platelet-monocyte conjugate, bleeding complications as well as inflammatory responses.

The anti-complement antibody can be monoclonal, polyclonal, monospecific, bispecific. The anti-complement antibody can be murine, mammalian, fully human, recombinant, chimeric, mutated or truncated. The anti-complement antibody can be a detection antibody or blocking antibody. The antigen binding fragments of the antibody can also be used. In essence, any peptide, protein, or amino acid motifs that can bind the complement protein of interest are within the scope of the present invention. Because variable regions of antibodies are conserved among IgG, F(ab)2, F(ab′)2, Fab′, Fab, scFv, recombinant, human, and truncated proteins—these can be immobilized onto the solid support to remove the complement proteins from body fluids.

The anti-complement antibody binding support structure can include a polymer matrix that has a substantial number of reactive groups, such as aldehyde, hydroxyl, thiol, carboxyl or amino groups, which can be activated for coupling the anti-complement antibody to the supportive structure. A polymer support matrix may include natural carbohydrates, such as agarose, cellulose or dextran or synthetic polymers including polystyrene, polyethersulfone, PVDF, ethylene vinyl alcohol, polycarbonate, polyether, polyether carbonate, regenerated cellulose, cellulose acetate, polylactic acid, nylon, or polyurethane. The physical shape of the matrix can be beads, fibers, tapes, filters.

The anti-complement antibody can be conjugated to the matrix by direct chemical linking, lipohillic moieties, or by other proteins known in the art. Proteins used to bind the anti-complement antibody to the matrix include proteins G, A, L and those that can mediate the binding of the matrix to the antibody without the loss of the antigen binding ability of the antibody to the proteins in fluid.

The antibody-bound matrix can be contained in a column or housing that allowS fluid flow. The material of the column can be an inert material. The column can have an inlet conduit and an outlet conduit and a fritted disc. The column can also withstand the flow rate commonly used in extracorporeal procedures. The column can also contain a valve to prevent the flash back of the forward moving blood.

The extracorporeal device, can be used alone (i.e., an extracorporeal circuit) to remove complement protein from blood before an extracorporeal circulation or procedure is begun. Removal of complement protein from the blood prior to an extracorporeal procedure such as cardio pulmonary bypass (CPB) is advantageous because the flow rate used for the extracorporeal device of the present invention may not be compatible with the flow rates observed during blood circulation in an extracorporeal CPB circuit.

The extracorporeal device can also be used concurrently with the extracorporeal circuit. Advantageously, the device should be connected between the patient and the extracorporeal circuit in such a way that blood from patient should flow into the device prior to coming in contact with the extracorporeal circuit.

In accordance with an aspect of the invention, the extracorporeal device can include a substrate-bound anti-properdin (i.e., anti-P) antibody, which removes properdin from the blood. The blood returning to the patient becomes devoid of properdin, which is critical for alternative complement pathway (i.e., AP) activation. In one example, an anti-P monoclonal antibody that specifically blocks the AP, can be immobilized onto large beads using protein-G coated sepharose B via chemical cross linking. The anti-P conjugated beads can then be incubated with whole human blood, plasma or serum. The anti-P conjugated beads incubated with the whole human blood, plasma, or serum can extract properdin by specific binding on properdin functional site and properdin will be removed from the blood, plasma, or sera. The extracorporeal device allows a sample of blood, plasma and sera to be safely rotated through an extracorporeal circuit with exposed foreign surfaces.

The beads to which the anti-P antibody is conjugated can be large enough to allow flow of cells through the extracorporeal device without shear. Because shear forces can cause cellular lysis, it is important to determine the size of beads appropriate for cells to pass through. CELLTHRUBIGBEADS (Sterogene Bioseparations, Inc., Carlsbad, Calif., USA) have been routinely used for such applications. The flow rate at which the blood will go through the device can be optimized to ensure consistency with the clinical application.

Anti-P can be conjugated directly to the beads but the binding efficiency of the anti-P may be reduced because of lack of proper orientation of the antibody onto the beads. Advantageously, a support matrix with reactive functional groups, such as, but not limited to, aldehyde, hydroxyl, thiol, amino or carboxyl groups, available for protein coupling can be used to promote anti-P antibody coupling to the matrix.

In one example, the anti-P can be immobilized by binding the anti-P to a cellulose support matrix, such as a regenerated cellulose hollow fiber membrane, which is used in hemodialyzers. Cellulose contains abundant hydroxyl groups, which can be activated with sodium metaperiodate, thus oxidizing them to aldehyde groups. Anti-P can be coupled to aldehyde groups of the cellulose by reductive amination. Aldehyde derivatization of other supports, such as polystyrene, polyethersulfone, PVDF, ethylene vinyl alcohol, polycarbonate, polyether, polyethercarbonate, polylactic acid, nylon, or polyurethane can be performed with formaldehyde or glutaraldehyde using standard chemical reactions (reviewed in Affinity Techniques, Methods in Enzymology Vol. 19A).

It has been shown that CELLTHRUBIGBEAD 300-500 micron beads, (Sterogene Bioseparations, Inc., Carlsbad, Calif., USA) allowed blood passage through a packed column Anti-P can be immobilized onto the novel aldehyde activated 4% agarose beads (300-500 micron particles)(e.g., LS Activated CELLTHRUBIGBEAD (Sterogene Bioseparations, Inc., Carlsbad, Calif., USA)) at 5 mg/ml following the manufacturer's directions. Other methods of making protein-conjugated columns have also been published.

Human anti-P derivatized 300-500 micron CELLTHRUBIGBEADs can be packed into 5 ml columns and perfused with 500 ml of fresh heparinized human blood. The flow rate that gives maximum retention of properdin and no shear of cells can then be determined. The ratio of volume of cartridge to the volume of blood can then be estimated based on the data generated. High flow rate is required in light of its use in a cardio pulmonary bypass (CPB) circuit. The effluents can be tested for red blood cell (RBC) hemolysis. The whole profile of cell differential can also be determined.

The device of this invention can be optimized for two different settings. In one setting, the device can be used without the CPB circuit being connected. In such case, a subject or patient can be connected to the device using catheters/tubing and a pump can be used to allow blood passage through the device. The effluent blood can be free of properdin where beads with anti-P coating is used. Cells in effluent blood should have the same profile as of the incoming blood (blood coming into the device). In optimizing this device, flow rate of blood does not have to match the flow rate used in CPB circuit.

In another setting, the device can also be used as a connector between the CPB circuit and the patient. Blood from the patient can flow into the device. The device can bind properdin in blood and pump the blood into the circuit. Such device would be ideal if flow rate of the device and CPB circuit is kept similar to avoid shear of RBCs and platelets.

Other extracorporeal settings can also use such a device since it is practically a complement protein removal system. Similar devices can also be made by coating the beads/matrix with anti-factor D, anti-factor C5 antibodies, anti-05a antibodies and others. In case of anti-05a, the device can also provide a means of continuously removing C5a from blood.

The extracorporeal device can also be used in the extracorporeal applications, such as dialysis, plasmapheresis, extracorporeal membrane oxygenation, hemodialysis, hemofiltration, open-heart surgery, and organ transplantation. Extracorporeal circulation is in part pathogenic because blood contact with the artificial circuits generates an inflammatory response and complement and other pathways relevant to blood are activated. Following the activation of complement, an intense cellular inflammatory response sets in causing a pathology, which leads to complications that arise following the extracorporeal circulation.

The main mechanism by which extracorporeal circulation of blood leads to morbidity and mortality is by producing anaphylatoxins C3a and C5a along with sC5b-9 complexes. Reduction in levels of C3a and C5a has been of great interest and such levels have been lowered by the drugs that prevent complement activation. While the drug may be effective, it becomes rather impossible to remove the drug following the extracorporeal circulation and the drug is removed via normal physiological route.

Previous studies have shown that antibodies and or small molecules to factor C3b, factor B, factor Bb, factor P, Factor C5, Factor D, Factor C6, C7, C8, and C9 can reduce the activation of complement activation. While activation of complement is controlled in such applications, the proteins remain in blood—attached to the drug antibody.

The present invention can thus remove such proteins from the body thereby preventing complement activation in blood during and following its contact with the artificial surfaces. All components C3b, factor B, factor Bb, factor P, Factor C5, Factor D, Factor C6, C7, C8, and C9 can be removed by affinity adsorption, utilizing as the adsorbent antibodies to these proteins or other specific chemical adsorbents, such as those that specifically bind the complement proteins.

Anti-complement antibodies bound to the matrix can remove specific proteins from blood because antibodies are known to be highly target specific. Several antibodies can be generated against a protein. Antibodies may be “detection or non-blocking” antibodies which detect the presence of the protein in a sample. Antibodies may be “blocking” antibodies, which bind to the protein at a specific site involved in function. For example, anti-C5 antibodies have been produced that prevent factor C5 cleavage into C5a and C5b (e.g., see PCT/US08/57468). Anti-P antibodies have been produced that prevent properdin binding to C3b (See PCT/US08/68530).

Both blocking and non-blocking antibodies can be used for removing proteins from a fluid. Both products can remove the specific protein from the fluid and therefore can be used to lower the concentration of the protein in fluid. There are major differences in these two approaches. When non-blocking antibodies are used for removing the specific protein, the functional site of the protein is not blocked and a fully functional protein remains bound to the matrix. When blocking antibodies are used for removing the specific protein, the functional site of the protein is blocked by the antibody to which the protein binds. The difference in the two approaches is highly significant with regards to generating an extracorporeal device. In the first approach, the device after coming in contact with the fluid will have the non-blocking antibody attached to the fully functional protein. For example, a non-blocking anti-properdin monoclonal antibodies when bound to a matrix in a device will remove properdin by binding to a non-active regions on properdin. In contrast, a blocking anti-properdin monoclonal antibodies when bound to the matrix in a device will remove properdin by binding to the functionally active regions on properdin. The use of a blocking anti-complement antibody is preferred because the complement protein is neutralized as it is being removed from circulation. A non-blocking properdin antibody, will remove properdin that while in the device will be capable of activating the complement pathway and could accumulate and participate in alternative pathway C3 convertase formation. Accordingly, the device of this invention can be prepared using blocking monoclonal antibodies especially those antibodies that are raised against intact, fragments, and fusion proteins derived from complement cascade.

The utilization of extracorporeal adsorption of properdin or any other complement protein the depletion of which can block the AP pathway is provided by this invention. Such adsorption can lead to marked reduction in the levels of that protein, thus would result in down regulation of the inflammatory responses during extracorporeal procedures. The device of the present invention thus provides a non invasive means of reducing AP activation in a human and has a significantly larger quantitative effect on additional factors that are involved in the etiology and pathogenesis of inflammation. For example, reduction of complement activation will also inhibit activation of cells that are part of the inflammatory cascade.

In accordance with another aspect of invention, the immuno-affinity adsorption of the antibody can be improved by immobilizing the antibody to the Staphylococcal Protein A. It will be appreciated that a recombinant Staphylococcal Protein A or Staphylococcal Protein A component, or other synthetic peptides of Staphylococcal Protein A may be utilized, as may Protein G or its components. Bensinger, U.S. Pat. No. 4,614,513; R. Lindmark et al., J. Immunological Methods, Vol. 62, 1983, p. 1. As used herein, except when the context clearly indicates otherwise, the terms “Protein A” and “Protein G” include all such variations.

When fragments of antibodies are used in the present invention as affinity adsorbents, they can be produced by enzymatic (e.g., papain or pepsin) digestion of the intact antibody to produce Fab, (Fab′)2, or FV antigen-binding fragments, or they can be produced by other methods known to those skilled in the art for the synthesis of peptides, such as solid phase synthesis (R. A. Houghten, Proc. National Academy of Science USA, Vol. 82, August 1985, pp. 5131-35; R. E. Bird et al., Science, Vol. 242, 1988, pp. 423-42). The use of fragments, rather than intact antibodies, as the affinity adsorbent may increase the adsorption capacity and reduce side effects that may be associated with the Fc non-antigen binding part of the antibody molecule.

### Example 1

### Anti-Complement Monoclonal Antibody for the Device

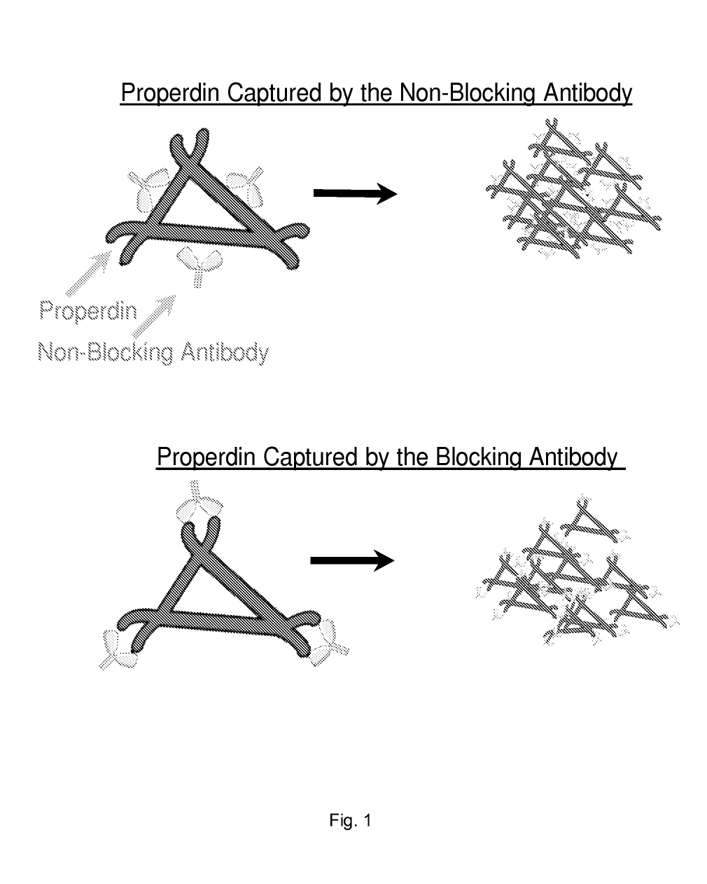

We selected an anti-P antibody that blocks the AP activation. The anti-P antibody is described in PCT/US08/68530. This antibody binds properdin and blocks properdin function. Properdin plays a role in AP activation and therefore, blockade of properdin function inhibits the AP. FIG. 1 shows a trimer of properdin monomer. Anti-P binds the TSR-1, which is represented in the Figure as corners of the trimer. Based on the molar ratio of anti-P to properdin, the model shown perfectly fits the anti-P used. This particular model also shows that if anti-P is immobilized onto the matrix and correctly oriented, it should bind the trimer and retain it onto itself. As a result, the blood/plasma samples passing through should become depleted of properdin. Since properdin plays a critical role AP, the blood and plasma should not activate the AP during blood transit through the extracorporeal circuit.

If non-blocking anti-P, which binds properdin but does not inhibit the AP activation is used, such antibodies will remove properdin from solution as shown by the binding of anti-P to the properdin trimer but the bound properdin would still be functional and would significantly activate the AP as more blood passes through the device. While properdin exists in all forms monomer, dimer, trimer, and tetramer, we will only be using the term trimer for convenience. The corners of the trimer will bind C3b and make an active C3 convertase in situ to allow AP activation to proceed. The device can then become a rich source of concentrated C3 convertase.

It is therefore important to develop a device where the anti-P is the one that blocks the AP and blocks the functionally important sites on the properdin molecule.

### Example 2

### Schematics of how the Anti-P Coated Bead Looks Like when Trimers of Properdin are Extracted from the Blood

Bead matrix (CELLTHRUBIGBEADS (Sreogene Corporation) or any bead with large diameter of nearly 300 microns) uncoated or coated with protein G is incubated with the anti-P monoclonal antibody to generate anti-P coated beads. Whole heparinized blood containing functional AP complement proteins is passed through the device. The anti-P coated onto the beads bind properdin from plasma. The flow through should have no AP activity.

### Example 3

### Schematics of Anti-P Coated Beads in a Column. View of Column Before and After Blood Passes Through. Absence of Properdin in Flow Through

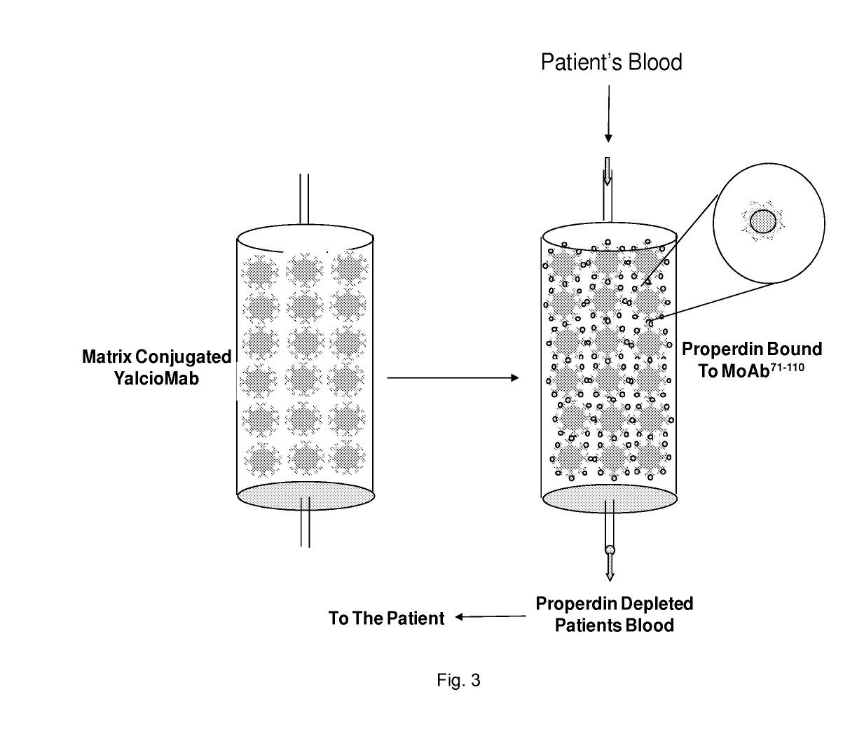

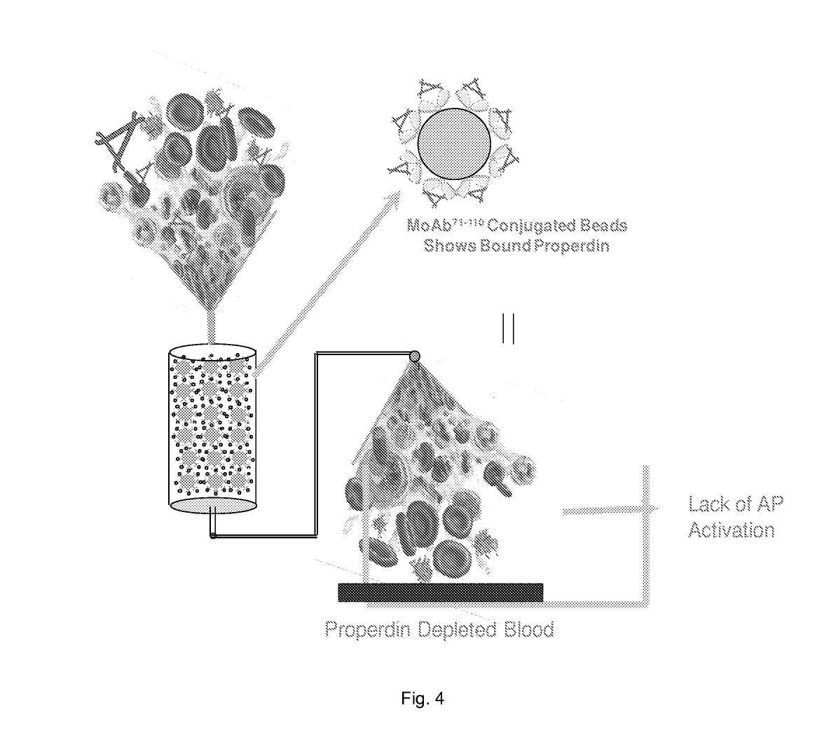

FIG. 3 illustrates two columns. The first column only has anti-P conjugated to the beads. The second column illustrates zoomed-out version of anti-P coated neads with retained properdin. An inset shows the zoom-in portion of the single bead with retained properdin. FIG. 4 shows that the blood that has been through the device is depleted off properdin. The outlet from the device is being poured into the container. The trimer triangles are missing from the flow through.

### Example 4

### Inhibition of AP Activation in Human Serum Depleted Off Properdin by MoAb⁷¹⁻¹¹⁰ Conjugated to Protein-G Coated Beads

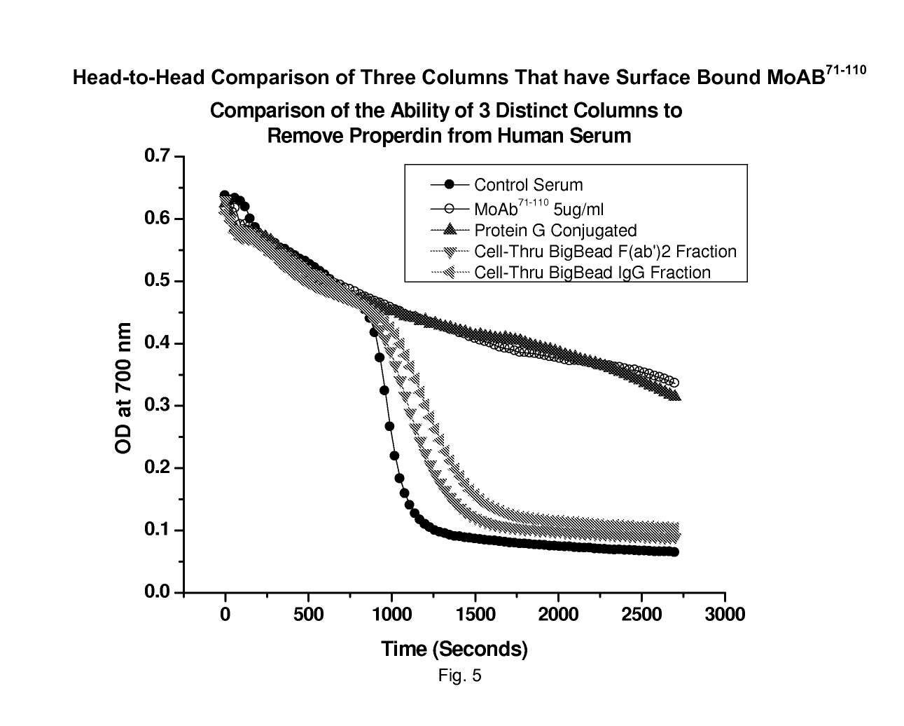

Three different columns were prepared; 1: CELLTHRUBIGBEADS (Sterogene Corporation) were chemically cross linked to the intact whole MoAb⁷¹⁻¹¹⁰, 2: CELLTHRUBIGBEADS were linked to F(ab′)2 of MoAb⁷¹⁻¹¹⁰, 3: Protein G Beads 40-160 microns covalently pre-coated with protein-G (Pierce chemical Co) were covalently linked to Whole MoAb71-110. These three matrices were used in columns of total capacity of 2 ml. These 2 ml columns were treated with 10 ml human serum and the pass through from the column was collected in a 15 ml tube. The 10 ml flow thru was combined with 40 ml AP buffer. The diluted serum pool was assayed by erythrocyte hemolysis assay. In a typical assay, 100 μl of the diluted serum was mixed with rabbit erythrocytes. The mixture was incubated at 37° C. in a temperature controlled ELISA plate. The lysis of cells was monitored at 700 nm over time. As shown the control serum shows complete lysis by the serum indicating AP activation. Both columns with CELLTHRUBIGBEADS also lysed the rabbit erythrocytes suggesting that human serum samples were nearly as potent as the untreated controls. The slight delay in lysis may only reflect dilution effect from the column and we conclude based on these data that both CELLTHRUBIGBEADS did not work. As expected, human serum with added MoAb⁷¹⁻¹¹⁰ antibody inhibits AP activation to baseline levels. As shown in FIG. 5, Column 3 (Protein-G conjugated monoclonal antibody) removed properdin and the properdin depleted serum loses AP activity in the hemolysis assay. These data suggest that substrate-bound (immobilized) MoAb⁷¹⁻¹¹⁰ is correct orientation is capable of binding properdin and removing it from human serum. Compared to the columns that had no protein-G coated, the flow thru serum had AP activity similar to controls.

### Example 5

### Removal of Properdin and Inhibition of AP Activity in Human Serum is Specific to the Monoclonal Antibody Conjugated Bead Matrix

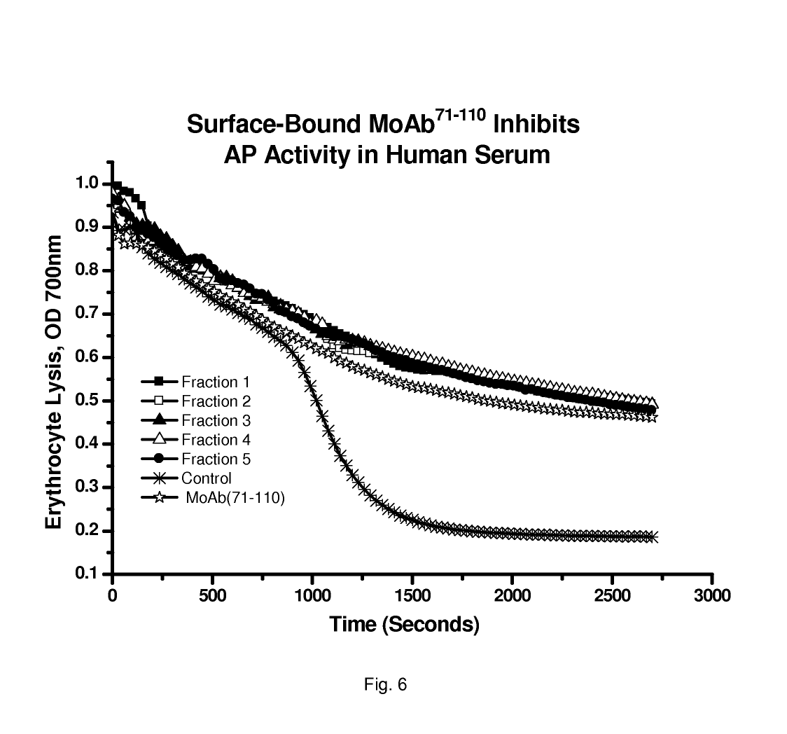

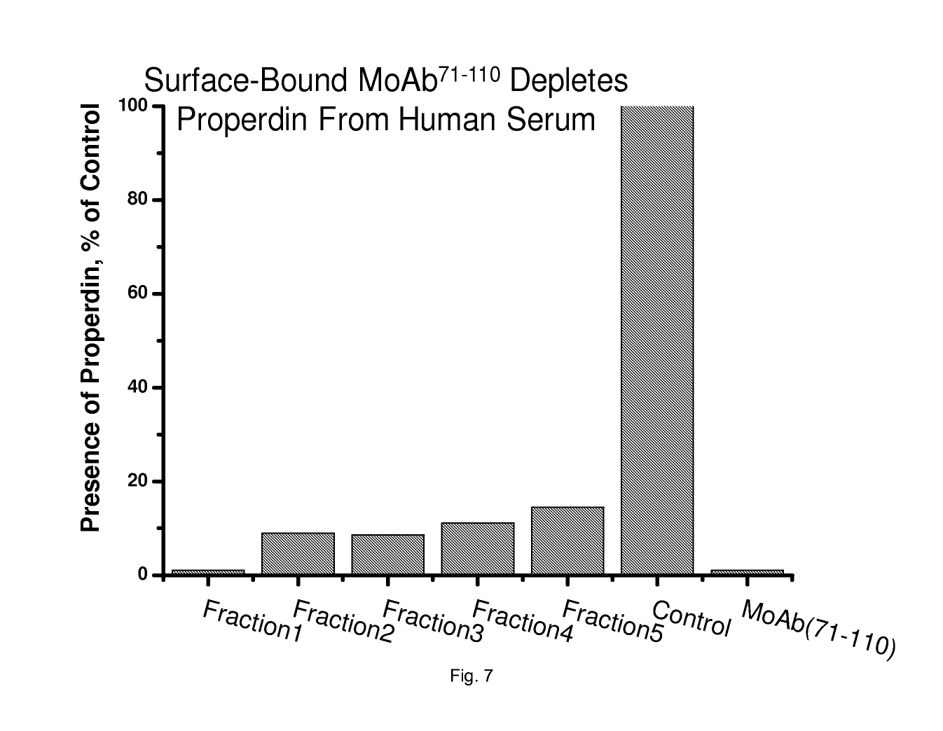

A column was prepared to evaluate the specificity of the substrate-bound monoclonal antibody on the protein-G matrix. Similar to example 4 above, the protein-G column was obtained from Thermo Scientific (Pierce Protein G IgG Plus Orientation Kit, cat# 44990). The monoclonal antibody was conjugated according to the manufacturer's instructions. The monoclonal antibody conjugated beads (2 ml) were placed in the polypropylene column. The column was washed phosphate buffer saline, pH 7.4. The human serum (5 ml) was placed on the column to allow its passage through the column. A total of five 1 ml fractions were collected. Each fraction was diluted with AP buffer and subjected to rabbit erythrocyte hemolysis assay. As shown in FIG. 6, all five fractions (1 ml each) inhibit AP activity in human serum. The serum control shows full complement activity. The cartridge shows that the substrate-bound monoclonal antibody to properdin would remove properdin from human serum thereby causing loss of hemolytic activity. All fractions and controls were also evaluated for the presence of properdin using an ELISA assay set up to quantitate properdin. In this properdin ELISA, the wells were coated with C3b (2 μg/50 μl/well) overnight. Next day the solution was removed and the wells were blocked with 1% BSA in PBS. Following the incubation at room temperature for 1 hour, aliquots of fractions were incubated with C3b coated wells. Properdin is known to bind C3b with high affinity. The presence of properdin was detected with anti-properdin antibody (P#2) from Quidel Corporation. This primary antibody was diluted 1:2000 in blocking buffer before application. The secondary antibody we used was an HRP-conjugated goat-antimouse monoclonal antibody. This antibody was also used at 1:2000 dilution. As shown in FIG. 7.

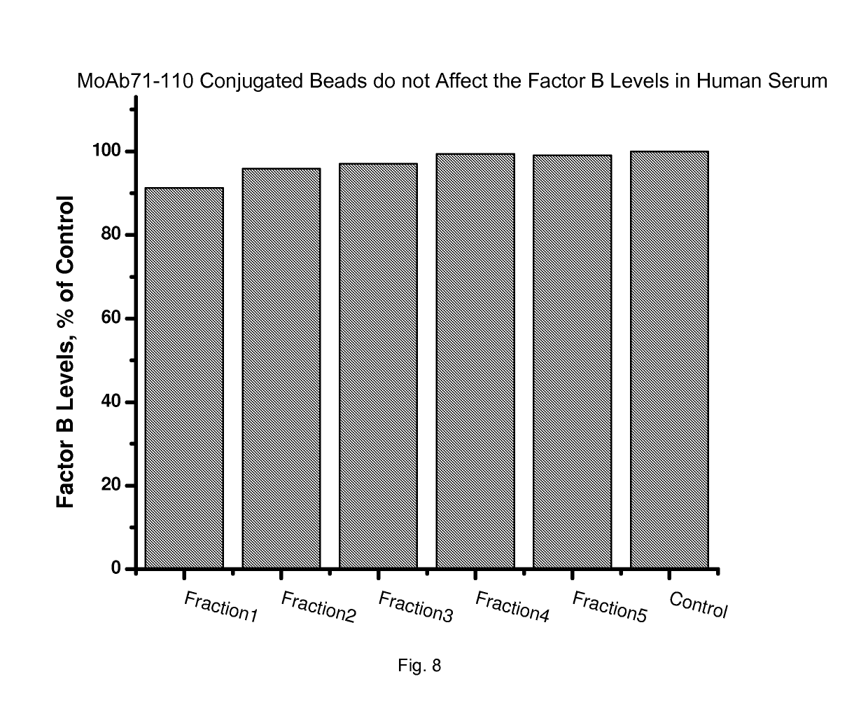

To determine if the loss of other proteins was contributing to the observed inhibition of AP activation, we measured the levels of factor B using an ELISA assay. In this assay, a monoclonal to factor B was coated onto the ELISA wells (2 μg/50 μl/well). Following blocking the wells were incubated with serum from various fractions. The bound factor B was detected with a polyclonal to factor B (American Qualex). The amount of factor B was determined using ordinary methods of detection. As shown in FIG. 8, levels of factor B were the same in all fractions suggesting no loss of factor B.

### Example 6

### Removal of Properdin and Inhibition of AP Activity in Human Serum is Specific to the Monoclonal Antibody Conjugated Bead Matrix

A column was prepared to evaluate the specificity of the substrate-bound monoclonal antibody on the protein-G matrix. Similar to example 4 above, the protein-G column was obtained from Thermo Scientific (Pierce Protein G IgG Plus Orientation Kit, cat# 44990). The monoclonal antibody was conjugated according to the manufacturer's instructions. The monoclonal antibody conjugated beads (2 ml) were placed in the polypropylene column. The column was washed phosphate buffer saline, pH 7.4. The human serum (5 ml) was placed on the column to allow its passage through the column. A total of five 1 ml fractions were collected. Each fraction was diluted with AP buffer and subjected to rabbit erythrocyte hemolysis assay. As shown in FIG. 6, all five fractions (1 ml each) inhibit AP activity in human serum. The serum control shows full complement activity. The cartridge shows that the substrate-bound monoclonal antibody to properdin would remove properdin from human serum thereby causing loss of hemolytic activity. All fractions and controls were also evaluated for the presence of properdin using an ELISA assay set up to quantitative properdin. In this properdin ELISA, the wells were coated with C3b (2 μg/50 μp/well) overnight. Next day the solution was removed and the wells were blocked with 1% BSA in PBS. Following the incubation at room temperature for 1 hour, aliquots of fractions were incubated with C3b coated wells. Properdin is known to bind C3b with high affinity. The presence of properdin was detected with anti-properdin antibody (P#2) from Quidel Corporation. This primary antibody was diluted 1:2000 in blocking buffer before application. The secondary antibody we used was an HRP-conjugated goat-antimouse monoclonal antibody. This antibody was also used at 1:2000 dilution. As shown in FIG. 7.

To determine if the loss of other proteins was contributing to the observed inhibition of AP activation, we measured the levels of factor B using an ELISA assay. In this assay, a monoclonal to factor B was coated onto the ELISA wells (2 μg/50 μl/well). Following blocking the wells were incubated with serum from various fractions. The bound factor B was detected with a polyclonal to factor B (American Qualex). The amount of factor B was determined using ordinary methods of detection. As shown in FIG. 8, levels of factor B were the same in all fractions suggesting no loss of factor B.

### Example 7

### Efficacy and Selectivity of the “Device” for Properdin Using Human Serum Flowing through the Monoclonal Antibody Column

We have repeatedly shown that monoclonal antibody conjugated to the bead matrix in the column is able to capture properdin and make the human serum depleted of the protein. It is concluded that because the antibody conjugated to the bead is an anti-properdin monoclonal antibody, properdin will bind the monoclonal antibody. Loss of properdin from human serum will result in loss of AP activity. However, it is sometimes possible that other proteins are also removed during this process. The most obvious one is factor D. It is possible that factor D is removed non-specifically from the serum. To determine the proteins being removed from the serum, we conducted another experiment to address two things 1) the efficacy of the 2 ml column—to determine the total serum it could safely produce without AP activity, 2) The specificity of the column to determine if it is only removing properdin.

The column was prepared as described in other examples. Human serum was passed through the column at a flow rate of 1 ml per minute. A total of 100 ml of serum was passed through the 2 ml column. A total of 100 fractions were collected in 1 ml volume. Each fraction was subjected to hemolysis assay for AP activity and for properdin assay to measure the amount of properdin. These two assays have already been described in examples above. Following the serum pass, the column was extensively rinsed with phosphate buffered saline. The column was eluted with an IgG Elution buffer (product # 21004) and the collected eluate was analyzed by western blot assay. A gradient 8-16% SDS-PAGE was run and the gel was transblotted onto a PVDF membrane using standard Western blotting techniques. One blot was probed with anti-properdin polyclonal antibody, the second one was probed with an anti-factor D polyclonal antibody and the third was probed without the primary antibody. All three blots were treated with a common secondary antibody—rabbit anti-goat polyclonal at 1:2000 dilution. Following the treatment, the bands were developed with DAB/Metal concentrate/Stable peroxide substrate system. A picture of the western blot is presented.

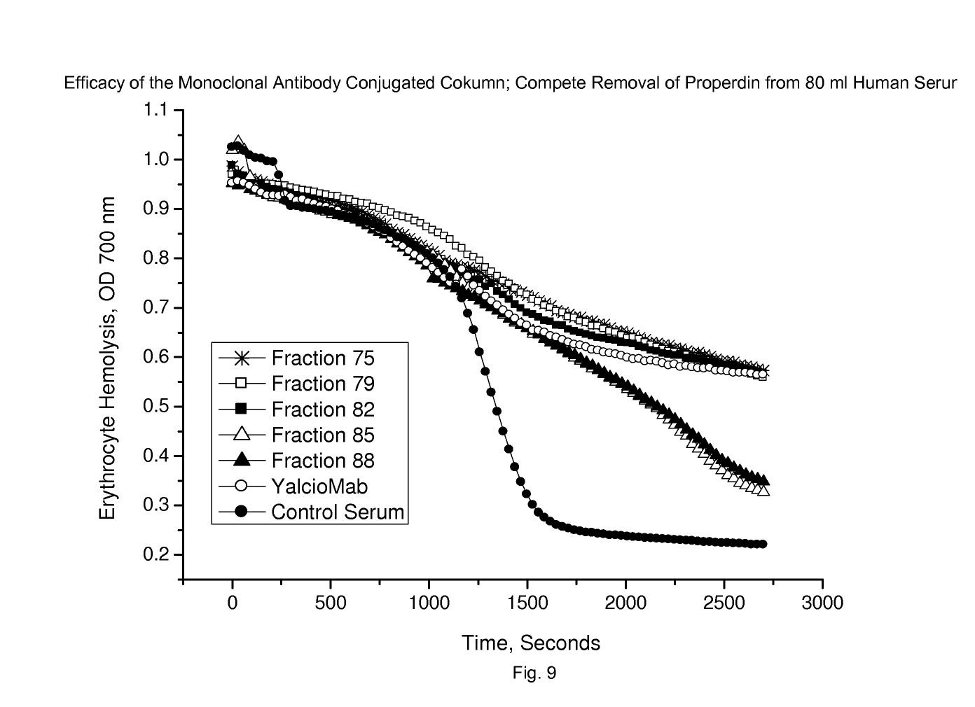

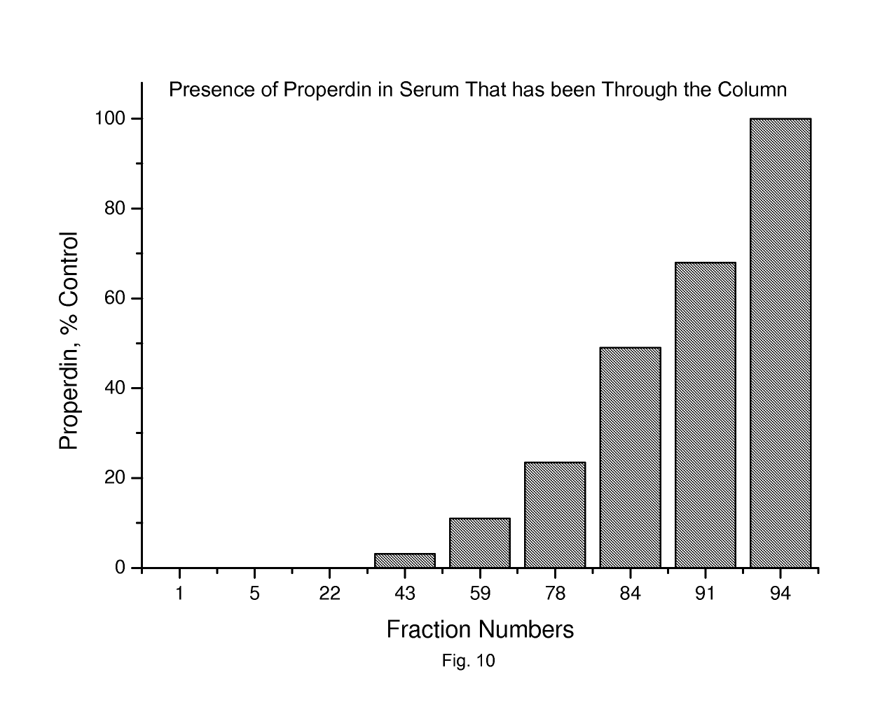

As shown in FIG. 9 fractions up through 82 completely inhibit the AP activation in human serum. Each fraction is of 1 ml, therefore the total volume of serum through the “device” is 82 ml. Fractions 85 through 88 show loss of AP inhibition suggesting that the “device” is incapable of retaining any extra properdin from human serum. The monoclonal antibody71-110 at 5 μg/ml of serum completely inhibits the AP activity showing that the fraction 82 and all before that inhibited AP activation in human serum. This data suggests that 2 ml of column is capable of retaining properdin from nearly 82 ml of total serum. Hypothetically using 40% as hematocrit average, we expect the total volume of blood to be nearly 140 ml of whole blood. Considering a 4500 ml of total blood in human body, 64 ml beads will be needed to completely block the AP activation in entire blood volume of 4500 ml. Fractions were also tested for the presence of properdin similar to those mentioned above. As shown in FIG. 10, properdin removal was complete in early fractions and the amount of properdin became visible in later fractions.

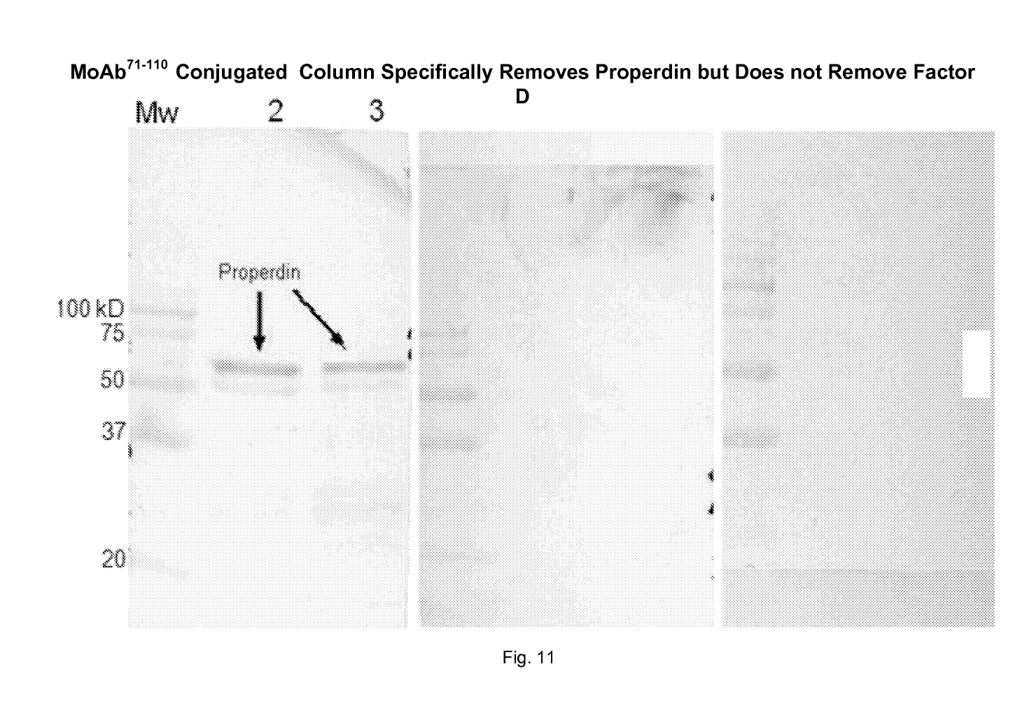

Shown in FIG. 11 are blots where properdin was identified using antibodies to properdin. In panel A is shown properdin, Panel B is from Factor D antibody probed blot and Panel is a control. As shown the eluted protein from the “Device” is the properdin and not factor B. the band shown is not an artifact of the secondary antibody as the panel C shows no bands.

### Example 8

### Efficacy of MoAb⁷¹⁻¹¹⁰ Binding to the Protein-G Coated Beads

In two column preparations, the efficiency of the beads capturing the monoclonal antibody was determined. As shown in data table-1, the first column efficiency was nearly 34% while the second time the efficiency is around 46%. These data show that monoclonal antibody can be chemically conjugated to the protein-G beaded matrix with full activity.

All references cited herein, including patents, patent applications, papers, textbooks, and the like, and the references cited therein, to the extent that they are not already, are hereby incorporated herein by reference in their entirety. In addition, the following references are also incorporated by reference herein in their entirety, including the references cited in such references.

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1-18. (canceled)

19. A method of inhibiting alternative complement pathway activation in a subject comprising:
passing a bodily fluid of the subject through an extracorporeal device, the device including a support structure, an anti-complement antibody disposed on or within the support structure, and a first conduit for conducting bodily fluid from the subject to the anti-complement antibody, the anti-complement antibody binding to and removing complement protein in the bodily fluid; and
returning the bodily fluid to the subject.

20. The method of claim 19, the bodily fluid comprising blood.

21. The method of claim 20, the anti-complement antibody inhibiting alternative complement pathway activation in the blood of subject.

22. The method of claim 19, wherein the support structure including a matrix that comprises at least one of agarose, cellulose, dextrin, polystyrene, polyethersulfone, polyvinyl difluoride, ethylene vinyl alcohol, polycarbonate, polyether, polyether carbonate, regenerated cellulose, cellulose acetate, polylactic acid, nylon, or polyurethane.

23. The method of claim 19, wherein the antibody comprises at least one of an anti-C3 antibody, anti-C3b antibody, anti-Ba antibody, anti-Bb antibody, anti-P antibody, anti-D antibody, anti-C5 antibody, anti-05a antibody, anti-C6 antibody, anti-C7 antibody, anti-C8 antibody, and anti-C9 antibody.

24-26. (canceled)

27. The method of claim 20, wherein the blood contacted with the anti-complement antibody is incapable of activating the alternative complement pathway when returned to the subject.

28. The method of claim 20, wherein the removal of the complement protein in the blood prevents activation of neutrophils, monocytes, basophils, lymphocytes, and platelets via the alternative pathway.

29. The method of claim 20, the anti-complement antibody being coated on the support structure.

30. The method of claim 20, the anti-complement antibody reduces the level of properdin in the blood.

31. The method of claim 30, the reduced levels of properdin in blood decreasing levels of C3a, C5a, Bb, C5b-9 as a result of decreased alternative complement pathway activation during extracorporeal circulation.

32. The method of claim 31, wherein the reduced levels of properdin reduces cellular activation in the subject following extracorporeal circulation.

33. The method of claim 20, the device further comprising a second conduit for returning blood to the subject, the complement protein being removed from the returned blood.

34. The method of claim 19, the anti-complement antibody being covalently adhered to a biocompatible polymer matrix.

35. The method of claim 34, wherein said polymer matrix is in the form of a membrane.

36. The method of claim 19, the support structure comprising a particulate polymer matrix, the particulate polymer matrix having reactive groups, wherein the reactive groups are selected from the group consisting of aldehyde, hydroxyl, thiol, carboxyl and amino groups.

37. An extracorporeal system for inhibiting alternative complement pathway activation in a subject, the system comprising:
a support structure,
an anti-complement antibody disposed on or within the support structure,
a first conduit for conducting blood of a subject to the anti-complement antibody, the anti-complement antibody binding to and removing complement protein in the blood,
a second conduit for returning blood contacted with anti-complement antibody to the subject.

38. The system of claim 37, the anti-complement antibody inhibiting alternative complement pathway activation in the blood of subject.

39. system of claim 37, wherein the support structure includes a matrix that comprises at least one of agarose, cellulose, dextrin, polystyrene, polyethersulfone, polyvinyl difluoride, ethylene vinyl alcohol, polycarbonate, polyether, polyether carbonate, regenerated cellulose, cellulose acetate, polylactic acid, nylon, or polyurethane.

40. system of claim 37, wherein the antibody comprises at least one of an anti-C3 antibody, anti-C3b antibody, anti-Ba antibody, anti-Bb antibody, anti-P antibody, anti-D antibody, anti-C5 antibody, anti-05a antibody, anti-C6 antibody, anti-C7 antibody, anti-C8 antibody, and anti-C9 antibody.

41. The system of claim 40, wherein the antibody is raised in a mammal.

42. The system of claim 40, wherein the antibody is monoclonal, polyclonal, recombinant, monospecific, bispecific, dimeric, humanized, chimeric, single chain, human, bispecific, truncated or mutated.

43. (canceled)

44. The system of claim 38, wherein the blood contacted with the anti-complement antibody is incapable of activating the alternative complement pathway when returned to the subject.

45. The system of claim 38, wherein the removal of the complement protein in the blood prevents activation of neutrophils, monocytes, basophils, lymphocytes, and platelets via the alternative pathway.

46. The system of claim 38, the anti-complement antibody being coated on the support structure.

47. The system of claim 37, the anti-complement antibody reduces the level of properdin in the blood.

48. The system of claim 47, the reduced levels of properdin in blood decreasing levels of C3a, C5a, Bb, C5b-9 as a result of decreased alternative complement pathway activation during extracorporeal circulation.

49. The system of claim 48, wherein the reduced levels of properdin reduces cellular activation in blood from the subject following extracorporeal circulation.

50. The system of claim 37, further comprising a second conduit for returning blood to the subject, the complement protein being removed from the returned blood.

51. The system of claim 37, the anti-complement antibody being covalently adhered to a biocompatible polymer matrix.

52. The system of claim 37, wherein said polymer matrix is in the form of a membrane.

53. The system of claim 37, the support structure comprising a particulate polymer matrix, the particulate polymer matrix having reactive groups, wherein the reactive groups are selected from the group consisting of aldehyde, hydroxyl, thiol, carboxyl and amino groups.

54. The system of claim 37, being coupled to at least one of an artificial heart-lung device or a hemodialysis unit such that blood flows through both the artificial heart lung device or hemodialysis unit and the system.
